(19) United States
(12) Patent Application Publication
(10) Pub. No.: US 2002/0192802 A1
(43) Pub. Date: Dec. 19, 2002

(54) Replication competent herpes virus strains

(76) Inventor: Robert Stuart Coffin, London (GB)

Correspondence Address: Nixon & Vanderhye, 8th Floor, 1100 North Glebe Road, Arlington, VA, 22201-4714, US

(21) Appl. No.: 10/181,698

(22) PCT Filed: Jan. 22, 2001
(86) PCT No.: PCT/GB01/00232

(30) Foreign Application Priority Data
Jan. 21, 2000 (GB) 0001476.1

(51) Int. Cl. A61K 48/00; A61K 39/245; A61K 39/255; A61K 39/265; A61K 39/27; C12N 7/00

(52) U.S. Cl. 435/235.1; 424/229.1; 424/93.2

(57) ABSTRACT

The present invention provides a herpes virus strain capable of replicating in permissive cells which comprises a mutation which results in enhanced ICP0 expression compared to the parental virus without said mutation for use in a method of treatment of the human or animal body by therapy.

### FIELD OF THE INVENTION

The present invention relates to herpes virus mutants with enhanced expression of ICP0. Such expression may be useful in (i) increasing expression of an inserted heterologous gene in target cells when an HSV strain is used as a vector and/or (ii) increasing the replicative ability of replication competent HSV strains in for example the oncolytic treatment of cancer.

### BACKGROUND TO THE INVENTION

Viruses have been suggested or demonstrated to have utility in a variety of applications in biotechnology and medicine on many occasions. Each is due to the unique ability of viruses to enter cells at high efficiency. This is followed in such applications by either virus gene expression and replication and/or expression of an inserted heterologous gene. Thus viruses can either deliver and express genes in cells (either viral or other genes) which may be useful in for example gene therapy or the development of vaccines, or they may be useful in selectively killing cells by lytic replication or the action of a delivered gene in for example cancer.

Herpes simplex virus (HSV) has been suggested to be of use both as a gene delivery vector in the nervous system and elsewhere and for the oncolytic treatment of cancer. In both applications the virus must however be disabled such that it is no longer pathogenic but such that it can still enter cells and perform the desired function. Thus for non-toxic gene delivery to target cells using HSV it has become apparent that in most cases immediate early gene expression must be prevented/minimised from the virus (Krisky et al 1998, Samaniego et al 1998). For the oncolytic treatment of cancer, which may also include the delivery of gene(s) enhancing the therapeutic effect, a number of mutations to HSV have been identified which still allow the virus to replicate in culture or in actively dividing cells in vivo (e.g. in tumors), but which prevent significant replication in normal tissue. Such mutations include disruption of the genes encoding ICP34.5, ICP6, and thymidine kinase. Of these, viruses with mutations to ICP34.5, or ICP34.5 together with mutation of e.g. ICP6 have so far shown the most favourable safety profile (see Andreansky et al 1996, Hunter et al 1999). Viruses deleted for only ICP34.5 have been shown to replicate in many tumor cell types in vitro and to selectively replicate in artificially induced brain tumors in mice while sparing surrounding tissue (e.g. see McKie et al 1996, Randazzo et al 1997). Early stage clinical trials have also shown their safety in man. However, such viruses may not show the maximum replicative potential in tumors of which HSV is capable. Thus viruses which still retain the safety of ICP34.5 mutated viruses, in that they do not replicate effectively in most non-tumor tissue in vivo, but which show enhanced oncolytic capabilties may improve the liklihood of success of HSV-based approaches to the oncolytic treatment of cancer.

### SUMMARY OF THE INVENTION

The present invention aims to provide viruses with enhanced oncolytic activity for the treatment of cancer as compared to those which were previously available by increasing the replicative abiltities of the virus in target cells.

The present invention provides a general means of enhancing gene expression from HSV which in the case of a replication competent virus can result in enhanced replication of the virus. The invention results from the finding that viruses mutated such that levels of expression of ICP0 are increased compared to otherwise identical viruses not mutated in such a manner, provide the required properties of enhanced replication. The present inventors have made the surprising finding that disabled HSV mutants with mutations in the region encoding the latency associated transcripts give enhanced levels of expression of ICP0. These viruses also replicate to a greater extent in permissive cells than do otherwise identical parental viruses. This was surprising as ICP0 expression levels would not be expected to be limiting in such circumstances.

ICP0 is an HSV immediate early gene with a promiscuous promoter activating capability which is important particularly in the early stages of the HSV infection cycle (e.g. Cai et al 1989). The invention therefore provides the opportunity to produce oncolytic HSV mutated for e.g. ICP34.5 with enhanced replicative and therefore oncolytic capabilities.

The invention also provides viruses which additionally carry a heterologous gene or genes. The term heterologous gene is intended to embrace any gene not found in the viral genome. The heterologous gene may be any allelic variant of a wild-type gene, or it may be a mutant gene. Heterologous genes are preferably operably linked to a control sequence permitting expression of said heterologous gene in a cell in vivo. Viruses of the invention may thus be used to deliver a heterotogous gene or genes to a cell in vivo where it will be expressed. For oncolytic virus therapy, such genes typically encode proteins capable of enhancing the tumor destroying properties of the virus. These genes may encode proteins which are themselves cytotoxic, are pro-drug activating, or which may be capable of stimulating/enhancing an anti-tumor immune response. In all cases single or multiple heterologous genes may be carried by a single virus.

Accordingly, the present invention provides:

a herpes virus strain mutated such that ICP0 expression levels are enhanced in permissive cells compared to the parental virus without said mutation, for use in a method of treatment of the human or animal body by therapy;

use of a virus strain according to the invention in the manufacture of a medicament for the treatment of cancer;

a method of treating a subject suffering from cancer, which method comprises administering a therapeutically effective amount of a virus strain; and

a pharmaceutical composition comprising, as active ingredient, a virus strain of the invention and a pharmaceutically acceptable carrier or diluent.

### DESCRIPTION OF THE FIGURES

FIG. 1(a) shows virus strains 17+/27−/w and 1764/27−/w which have been previously described in Thomas et al., 1999.

FIG. 1(b) shows the pR191acZ cassette and illustrates the site of insertion of the CMV/lacZ/polyA cassette in the LAT region.

### DETAILED DESCRIPTION OF THE INVENTION

Viruses

Although the present invention has been exemplified using herpes simplex viruses, it will be understood that other viruses of the herpesviridae family may be modified such that they show enhanced replication in permissive cells by the inclusion of mutations resulting in increased expression of the ICP0-homologue protein. In particular, such viruses may include varicella zoster virus, pseudo-rabies virus or bovine herpes viruses.

When the virus of the invention is a herpes simplex virus, the virus may be derived from, for example, HSV1 or HSV2 strains, or derivatives thereof, preferably HSV1. Derivatives include inter-type recombinants containing DNA from HSV1 and HSV2 strains. Such inter-type recombinants are described in the art, for example in Thompson et al (1988) and Meignier et al (1988). Derivatives preferably have at least 70% sequence homology to either the HSV1 or HSV2 genomes, more preferably at least 80%, even more preferably at least 90 or 95%. More preferably, a derivative has at least 70% sequence identity to either the HSV1 or HSV2 genome, more preferably at least 80% identity, even more preferably at least 90%, 95% or 98% identity.

For example the UWGCG Package provides the BESTFIT program which can be used to calculate homology (for example used on its default settings) (Devereux et al. (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al. (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al., 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value: the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4. and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

A derivative may have the sequence of a HSV1 or HSV2 genome modified by nucleotide substitutions, for example from 1, 2 or 3 to 10, 25, 50 or 100 substitutions. The HSV1 or HSV2 genome may alternatively or additionally be modified by one or more insertions and/or deletions and/or by an extension at either or both ends.

Viruses of the invention are produced by mutating an HSV strain such that the mutation results in enhanced levels of expression of ICP0 as compared to the parental virus. This may be accomplished in a number of ways:

A mutation may be made to the endogenous latency associated transcript (LAT) regions of the virus resulting in increased ICP0 expression in at least two ways. This can be by disrupting the LAT region such that the RNA which is usually generated which is antisense to the RNA encoding ICP0 (Stevens et al 1987) is either no longer produced, or so that expression levels are reduced, or so that an RNA with an altered antisense region to the ICP0-RNA is produced. Alternatively a sequence ; which may increase transcription of the ICP0 RNA may be inserted, for example an enhancer sequence such as from the CMV IE promoter may be inserted into the LAT region giving such a result. The LAT region is defined as the region within the long terminal repeats of HSV between the unique long region and the 5′terminus of the LAT RNA transcript approximately 8 kb. For example, in HSV1 strain 17+(GenBank accession no. HE1CG) one LAT region is the region from the start of the internal long repeat region at nucleotide 117,160 to the polyA site for the LAT RNA transcript of approximately 8 kb at nucleotide 127,142 and the second LAT region is the equivalent region in the terminal long repeat region. In other strains of HSV1 and in strains of HSV2, one LAT region is between nucleotides corresponding to 117,160 and 127,142 and the second LAT region is between the equivalent nucleotides in the terminal long repeat region. For example, in HSV2 strain HG52 (GenBank accession no. NC001798) one LAT region is the region from the start of the internal long repeat region at nucleotide 117,987 to the polyA site for the LAT RNA transcript of approximately 8 kb at nucleotide 127,915 and the second LAT region is the equivalent region in the terminal long repeat region.

A second means by which ICP0 expression levels may be increased would be by making alterations to the ICP0 promoter, or by replacing the endogenous ICP0 promoter with a promoter resulting in enhanced transcription of ICP0-encoding sequences in target cells. Similarly an ICP0-encoding gene may be inserted at an exogenous position in the HSV genome, under appropriate promoter control, resulting in enhanced expression levels of ICP0, i.e. an ICP0-encoding sequence operably linked to a promoter is here inserted into the HSV genome (either with or without accompanying deletion of the endogenous ICP0-encoding genes) away from the usual ICP0-encoding locus. A gene encoding a non-HSV homologue of ICP0 may also be inserted into the HSV genome giving a similar replication-enhanced phenotype to the resulting virus, or another gene with a similar generalised transcription enhancing property used. Non-HSV homologues of ICP0 include proteins with similar functions from other herpes viruses, such as ORF61 from varicella zoster virus, pseudo rabies EPO protein and BICP0 from bovine herpes virus.

Alterations to the ICP0 promoter that enhance promoter activity may be made. For example, an enhancer sequence may be inserted into the region of the virus genome upstream of or downstream from the promoter such that promoter activity is enhanced. Alternatively the promoter may be mutated such that transcription factor binding activity is increased. Any inhibiting sequences which affect promoter activity may also be disrupted, for example by deletion or substitution.

The ICP0 promoter may be replaced by any suitable promoter that has a higher activity in tumour cells than the endogenous ICP0 promoter. For example, the ICP0 gene may be operably linked to any promoter which is functional in mammalian, preferably human, tumour cells. The promoter may be derived from a promoter sequences of a eukaryotic gene. For example, the promoter may be derived from the genome of a cell in which expression of the ICP0 gene is to occur, preferably a mammalian, preferably human tumour cell. The promoter may function in a ubiquitous manner (such as promoters of β-actin or tubulin) or, alternatively, in a tissue-specific manner. It may also be promoter that responds to a specific stimulus, for example it may bind a steroid hormone receptor. A viral promoter may also be used, for example the Moloney murine leukaemia virus long terminal repeat (MMLV LTR) promoter or another retroviral promoter or the human or mouse cytomegalovirus (CMV) IE promoter.

ICP0 expression is usually stimulated from the HSV genome by the virion protein vmw65, and therefore alterations to vmw65 expression, or insertion of genes encoding non-HSV homologues of vmw65 (such as from equine herpes virus [EHV] or bovine herpes virus [BHV]; see Thomas et al 1999) may result in a similar phenotype in the resulting virus as in the preceding embodiments above.

All of the above methods, which are intended to be illustrative and not limiting, may result in a virus with enhanced levels of ICP0 expression and thus enhanced replicative capabilities in permissive cells.

ICP0 expression may be monitored by any suitable technique. For example, levels of ICP0 mRNA may be monitored by northern blotting and/or ICP0 protein may be monitored using western blotting techniques. Preferably levels of ICP0 expression by a virus of the invention in permissive cells, such as B130/2 cells, is at least 1.1 fold, preferably 1.2 fold, 1.3 fold, 1.5 fold, 2 fold, 5 fold, 10 fold, 20 fold, 50 fold or 100 fold greater than by a parental strain.

In a particularly preferred embodiment of the present invention, a disruption is made to the endogenous LAT regions of a strain of HSV from which a functional ICP34.5 protein cannot be expressed. Disruption of the LAT region results in increased expression of ICP0 in a target tumor cell, and other permissive cells for example B130/2 cells, such that replication, and thus oncolytic activity, is enhanced.

In a further preferred embodiment, the virus of the invention also carries a heterologous gene encoding a protein capable of modifying an immune response such as GM-CSF or other cytokine, a chemokine such as RANTES or other immunomodulatory molecule such as B7.1, B7.2 or CD40L. A virus of the invention may also contain a mutation preventing the expression of a functional ICP47 protein. ICP47 is an HSV IE gene which functions to reduce the antigen presenting capabilities of HSV infected cells and which may thus reduce the anti-tumor immune effects of oncolytic HSV.

Viral regions altered for the purposes of attenuation in such viruses (i.e. mutations which allow replication in tumor cells but not in non-tumor tissue) may be either eliminated (completely or partly), or made non-functional, or substituted by other sequences, in particular by a heterologous gene sequence. Such regions in HSV include mutation to the genes encoding ICP34.5, ICP6 or thymidine kinase. In a particularly preferred embodiment the gene encoding ICP34.5 is mutated such that an active form of ICP34.5 cannot be produced.

Viruses of the invention are modified oncolytic viruses. Such viruses infect and replicate in tumor cells, subsequently killing the tumor cells. Thus, such viruses are replication competent. Preferably, they are selectively replication competent in tumour cells. This means that they replicate in tumour cells and not in non-tumour cells than in non-tumour cells. Cells in which the virus is able to replicate are permissive cells. Examples of permissive cells which may be used to test the levels of the ICP0 expression and cell-killing capacity of a virus of the invention include B130/2 cells and tumour cell lines such as HT29 (colorectal adenocarcinoma), LNCaP.FGC (prostate adenocarcinoma), MDA-MB-231 (breast adenocarcinoma), SK-MEL-28 (malignant melanoma) and U-87 MG (glioblastoma astrocytoma). Cells in which the virus is not able to replicate or can replicate only inefficiently are non-permissive cells. BHK cells are an example of non-permissive cells. Measurement of selective replication competence can be carried out by the tests described herein for measurement of replication and tumour cell-killing capacity, and may also be analysed by the statistical techniques mentioned herein.

An oncolytic virus of the invention preferably has a greater ability than an unmodified parent strain to infect or replicate in a tumor cell, to kill tumor cells or to spread between cells in tissues. Preferably, this ability is a statistically significantly greater ability. Preferably, a virus of the invention has a greater ability, as measured by standard statistical tests, than an unmodified parent strain to infect or replicate in any tumor cell, to kill tumor cells or to spread between cells in tissue. More preferably, such greater ability is a statistically significant greater ability. For example, according to the invention, a may have up to 1.1 fold, 1.2 fold, 1.5 fold, 2 fold, 5 fold, 10 fold, 20 fold, 50 fold, or 100 fold the capacity of the parent strain in respect of the property being tested.

The properties of the virus strain in respect of tumor cells can be measured in any manner known in the art. For example, the capacity of a virus to infect a tumor cell can be quantified by measuring the dose of virus required to measure a given percentage of cells, for example 50% or 80% of cells. The capacity to replicate in a tumor cell can be measured by growth measurements such as those carried out in the Examples, e.g. by measuring virus growth in cells over a period of 6, 12, 94, 36, 48 or 72 hours or longer.

The ability of a virus to kill tumor cells can be roughly quantitated by eye or more exactly quantitated by counting the number of live cells that remain over time for a given time point and MOI for given cell type. For example, comparisons may be made over 24, 48 or 72 hours and using any known tumor cell type. In particular, HT29 colorectal adenocarcinoma, LNCaP.FGC prostate adenocarcinoma, MDA-MB-231 breast adenocarcinoma, SK-MEL-28 malignant melanoma or U-87 MG glioblastoma astrocytoma cells can be used. Any one of these cell types or any combination of these cell types can be used, as may other tumor cell types. It may be desirable to construct a standard panel of tumor cell types for this purpose. To count the number of live cells remaining at a given time point, the number of Trypan blue-excluding cells (i.e. live cells) can be counted. Quantitation may also be carried out by fluorescence activated cell sorting (FACS) or MTT assay. Tumor cell-killing ability may also be measured in vivo, e.g. by measuring the reduction in tumor volume engendered by a particular virus.

The ability of a virus to spread in tissue, especially solid tissue, can be measured by determining the number of cells at sites connected to the site of the original infection.

Methods of Mutation

The various disruptions and alterations of the herpes virus genome referred to herein may be made using any of several techniques well known in the art. For example, deletion(s), substitution(s) or insertion(s), preferably deletion(s) may be made to the viral genome in the specified region(s). A deletion may remove portions of the region or the entire region. Similarly, a substitution may replace a portion of the region or the entire region. For example deletion or substitution of only one nucleotide may be made. However, preferably larger deletions or substitutions are made, for example from 2, 3 or 5 to 10, 20, 30. 50. 100 or 200 nucleotide substitutions. Preferably at least 25%, more preferably at least 50% of the total coding and non-coding sequence (or alternatively, in absolute terms, at least 10 nucleotides, more preferably at least 100 nucleotides, most preferably, at least 1000 nucleotides) is deleted or substituted. Heterologous gene/genes may be inserted either into or replacing the region to be disrupted, or alternatively at alternative site/sites. For example, in a virus in which the LAT region is disrupted, a heterologous gene may be inserted into the LAT region.

The various viral genes referred to may be rendered functionally inactive by several techniques well known in the art. For example, they may be rendered functionally inactive by one or more deletion, substitution or insertion, preferably by deletion. Deletions may remove portions of the genes or the entire gene. For example, deletion of only one nucleotide may be made resulting in a frame shift. However, preferably larger deletions are made, for example at least 25%, more preferably at least 50% of the total coding and non-coding sequence (or alternatively, in absolute terms, at least 10 nucleotides, more preferably at least 100 nucleotides, most preferably at least 1000 nucleotides). It is particularly preferred to remove the entire gene and some of the flanking sequences. Heterologous gene/genes may be inserted either into or replacing the gene to be rendered functionally inactive, or alternatively at alternative site/sites/. For example, in a virus in which ICP34.5 is functionally inactive, a heterologous gene may be inserted into the ICP34.5 encoding region itself or elsewhere in the genome.

Mutations are made in the herpes viruses by homologous recombination methods well known to those skilled in the art. For example, HSV genomic DNA is transfected together with a vector, preferably a plasmid vector, comprising the mutated sequence flanked by homologous HSV sequences. The mutated sequence may comprise deletions, insertions or substitutions, all of which may be constructed by routine techniques. Insertions may include selectable marker genes, for example lacZ or GFP, for screening recombinant viruses by, for example, β-galactosidase activity or fluorescence.

Heterologous Genes and Promoters

The viruses of the invention may be modified to carry a heterologous gene/genes. The term “heterologous gene” encompasses any gene. The heterologous gene typically encodes a protein capable of enhancing the anti-tumour activity of the virus. Preferably the heterologous gene encodes an immunomodulatory protein such as GM-CSF or other cytokine, a chemokine such as RANTES or another immunomodulatory molecule such as B7.1, B7.2 or CD40L. Heterologous genes may alternatively encode proteins which are themselves cytotoxic or may encode pro-drug activating enzymes.

Although a heterologous gene is typically a gene not present in the genome of a herpes virus, herpes gene/genes may be used provided that the coding sequence is not operably linked to the viral control sequences with which it is naturally associated. The heterologous gene may be any allelic variant of a wild-type gene, or it may be a mutant gene. A virus of the invention may optionally include some or all of 5′ and/or 3′ transcribed but untranslated flanking sequences naturally, or otherwise, associated with the translated coding sequence of a heterologous gene. It may optionally further include the associated transcriptional control sequences normally associated with the transcribed sequences, for example transcriptional stop signals, polyadenylation sites and downstream enhancer elements.

The heterologous gene/genes may be inserted into the viral genome by homologous recombination of HSV strains with, for example, plasmid vector(s) carrying the heterologous gene/genes flanked by HSV sequences. The heterologous gene/genes may be introduced into a suitable plasmid vector comprising herpes viral sequences using cloning techniques well-known in the art. The heterologous gene/genes may be inserted into the viral (genome at any location provided that the virus can still be propagated and such that oncolytic potential is not diminished. Heterologous genes may be inserted at multiple sites within the virus genome.

The transcribed sequence of the heterologous gene/genes is preferably operably linked to a control sequence permitting expression of the heterologous gene/genes in mammalian cells, preferably a tumor cell, more preferably a human tumour cell. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence.

The control sequence comprises a promoter allowing expression of the heterologous gene/genes and a signal for termination of transcription. The promoter is selected from promoters which are functional in mammalian, preferably human, tumor cells. The promoter/promoters may be derived from promoter sequences of eukaryotic genes. For example, promoters may be derived from the genome of a cell in which expression of the heterologous gene is to occur, preferably a mammalian, preferably human tumor cell. With respect to eukaryotic promoters, they may be promoters that function in a ubiquitous manner (such as promoters of β-actin. tubulin) or, alternatively, a tissue-specific manner. They may also be promoters that respond to specific stimuli, for example promoters that bind steroid hormone receptors. Viral promoters may also be used, for example the Moloney murine leukaemia virus long terminal repeat (MMLV LTR) promoter or other retroviral promoters, the human or mouse cytomegalovirus (CMV) IE promoter, or promoters of herpes virus genes including those driving expression of the latency associated transcripts.

Expression cassettes and other suitable constructs comprising the heterologous gene/genes and control sequences can be made using routine cloning techniques known to persons skilled in the art (see, for example, Sambrook, 1989, Molecular Cloning—A laboratory manual; Cold Spring Harbor Press).

It may also be advantageous for the promoters to be inducible so that the levels of expression of the heterologous gene can be regulated. Inducible means that the levels of expression obtained using the promoter can be regulated. For example, in a preferred embodiment where more than one heterologous gene is inserted into the HSV genome, one promoter would comprise a promoter responsive to the tet repressor/VP16 transcriptional activator fusion protein previously reported (Gossen and Bujard, 1992, Gossen, 1995), and driving the heterologous gene the expression of which is to be regulated. The second promoter would comprise a strong promoter (e.g. the CMV IE promoter) driving the expression of the tet repressor/VP16 fusion protein. Thus in this example expression of the first heterologous gene would depend on the presence or absence of tetracycline.

Heterologous genes may also include marker genes (for example β-galactosidase or green fluorescent protein or other fluorescent proteins) or genes whose products regulate the expression of other genes (for example, transcriptional regulatory factors including the tet repressor/vmw65 transcriptional activator fusion protein described above).

Cancer therapy may well require the administration of multiple genes. The expression of multiple genes may be advantageous for the treatment of a variety of conditions. Herpes viruses are uniquely appropriate as they do not have the limited packaging capabilities of other viral vector systems. Thus multiple heterologous genes can be accommodated within its genome. For example, from 2 to 5 genes may be inserted into the genome.

There are, for example, at least two ways in which this could be achieved. For example, more than one heterologous gene and associated control sequences could be introduced into a particular HSV strain either at a single site or at multiple sites in the virus genome. It would also be possible to use pairs of promoters (the same or different promoters) facing in opposite orientations away from each other, these promoters each driving the expression of a heterologous gene (the same or different heterologous gene) as described above.

Therapeutic Uses

Viruses of the invention may be used in methods of therapy of the human or animal body. In particular, viruses of the invention may be used in the oncolytic treatment of cancer, either with or without additional pro-drug therapy or stimulation of an immune response. Viruses of the invention may be used in the therapeutic treatment of any solid tumour in a mammal, preferably in a human. For example viruses of the invention may be administered to a subject with prostate, breast, lung, liver, endometrial, bladder, colon or cervical carcinoma; adenocarcinoma; melanoma; lymphoma; glioma; or sarcomas such as soft tissue and bone sarcomas.

Administration

The viruses of the invention may be used in a patient, preferably a human patient, in need of treatment. A patient in need of treatment is an individual suffering from cancer, preferably an individual with a solid tumour. The aim of therapeutic treatment is to improve the condition of a patient. Typically therapeutic treatment using a virus of the invention allieviates the symptoms of the cancer. A method of treatment of cancer according to the invention comprises administering a therapeutically effective amount of a virus of the invention to a patient suffering from cancer. Administration of an oncolytic virus of the invention to an individual suffering from a tumour will typically kill the cells of the tumour thus decreasing the size of the tumour and/or preventing spread of malignant cells from the tumour.

One method of administering therapy involves combining the virus with a pharmaceutically acceptable carrier or diluent to produce a pharmaceutical composition. Suitable carriers and diluents include isotonic saline solutions, for example phosphate-buffered saline.

Treatment may then be carried out following direct injection of the virus composition into target tissue which may be the tumour or a blood vessel supplying the tumour. The amount of virus administered is in the case of HSV in the range of from 10⁴ to 10¹⁰ pfu, preferably from 10⁵ to 10⁸ pfu, more preferably about 10⁶ to 10⁸ pfu. Typically up to 500 μl, typically from 1 to 200 μl preferably from 1 to 10 μl of a pharmaceutical composition consisting essentially of the virus and a pharmaceutically acceptable suitable carrier or diluent would be used for injection. However for some oncolytic therapy applications larger volumes up to 10 ml may also be used, depending on the tumour and the inoculation site.

The routes of administration and dosages described are intended only as a guide since a skilled practitioner will be able to determine readily the optimum route of administration and dosage. The dosage may be determined according to various parameters, especially according to the location of the tumour, the size of the tumour, the age, weight and condition of the patient to be treated and the route of administration. Preferably the virus is administered by direct injection into the tumour. The virus may also be administered systemically or by injection into a blood vessel supplying the tumour. The optimum route of administration will depend on the location and size of the tumour.

The following Example illustrates the invention.

Materials and Methods

Virus Strains

Virus strains used are mutated for ICP27 (virus strains strains 17+/27−/w, 17+/27−/pR191acZ) or ICP27, ICP34.5 and vmw65 (virus strains 1764/27−/w and 1764/27−/pR191acZ) and are derived from HSV1 strain 17+ the nucleotide sequence of which is deposited in GenBank (Accession No. HE1CG). These virus strains can be propagated on B130/2 cells, either with or without HMBA (MacFarlane et al 1992; which improves growth of viruses with mutations in vmw65). B130/2 cells complement mutations to ICP27 (Howard et al 1998).

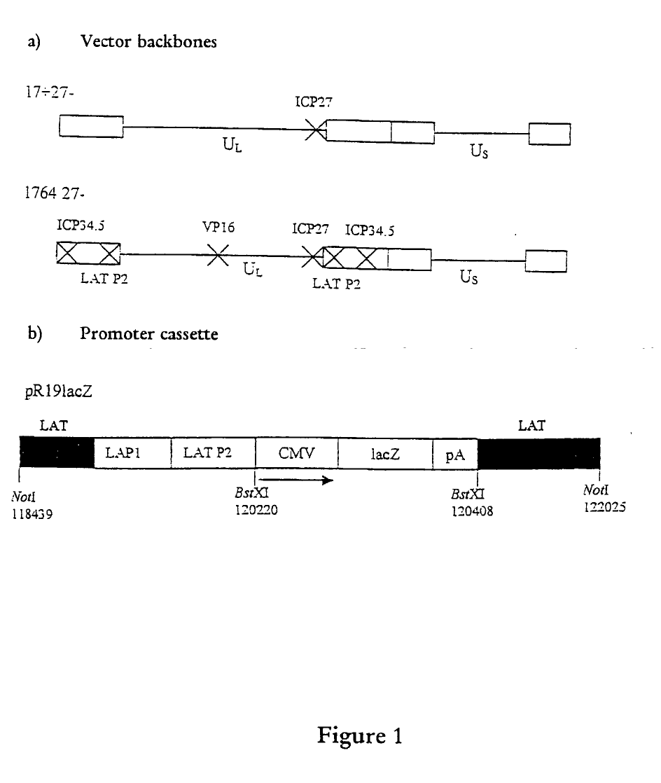

Virus strains 17+/27−/w and 1764/27−/w have been previously described (Thomas et al 1999) and are shown in FIG. 1(a). The term 17+ refers to HSV strain 17+. The term 1764 describes a virus with the in 1814 mutation in the gene encoding VMW65 and with the genes encoding ICP34.5 and ORF P completely deleted (between nucleotides nt 124945-125723). The term 27− refers to the deletion of nucleotides 113273-116869, which contains the genes UL54, 55 and 56. UL54 is the gene encoding the essential IE gene ICP27 and UL55 and 56 are both nonessential genes.

Virus strains 17+/27−/pR191acZ, and 1764/27−/pR191acZ contain a CMV promoter (from pcDNA3; Invitrogen)/lacZ (pCH110; Pharmacia)/poly A (from pcDNA3) cassette inserted into both copies of the LAT region between the BstXI sites at nts 120,219 and 120,413 in virus strains 17+/27−/w and 1764/27−/w respectively. FIG. 1(b) shows the pR191acZ cassette and illustrates the site of insertion of the CMV/lacZ/polyA cassette into the LAT region. Virus strain 17+/27−/pR191acZ has been previously described (Wagstaff et al 1998).

### Example 1

### HSV Mutants in Which the LAT Region Has Been Disrupted Show Enhanced Expression of ICP0 and Enhanced Replication in Permissive Cells

(i) Western blots were performed in which ICP0 levels were compared following inoculation of BHK cells with each of the viruses 17+/27−/w, 17+/27−/pR191acZ, 1764/27−/w or 1764/27−/pR191acZ at a multiplicity of infection (MOI) of 5 and harvesting cells 24 hrs later. BHK cells are non-permissive for the growth of these viruses as they do not express the essential ICP27 gene which has been deleted from the virus and thus any differences detected are due to the differing expression levels of ICP0 rather than other factors such as differential virus growth. These blots showed that while ICP0 could easily be detected using an anti-ICP0 antibody in each case, virus strains 17+/27−/pR191acZ and 1764/27−/pR191acZ showed enhanced ICP0 expression levels as compared to 17+/27−/w or 1764/27−/w.

(ii) Growth curves were performed in which B130/2 cells (which are permissive for the growth of HSV deleted for ICP27, including HMBA for the growth of 1764 viruses) were inoculated with each of the viruses 17+/27−/w, 17+/27−/pR191acZ, 1764/27−/w or 1764/27−/pR191acZ at an MOI of 0.01 and samples from the cells harvested 6, 16, 24 and 36 hrs later. Samples were then titrated onto fresh B130/2 cells (including HMBA for 1764 viruses) allowing an assessment of virus growth. These titrations showed that disruption of the LAT region in 17+/27−/pR191acZ and 1764/27−/pR191acZ had provided viruses which generated a titre under the conditions described which was 5-10 fold higher than was achieved with the parental 17+/27−/w and 1764/27−/w viruses after 36 hrs growth.

Therefore with two different virus mutants, disruptions to the LAT region of the virus result in enhanced expression of ICP0, which in turn results in increased replication of the virus in permissive cells.

### REFERENCES

Cai et al 1989 J. Virol 63; 4579-4589

Randazzo et al 1997 J Invest Dermatol 108; 933-937

McKie et al 1996 Br J Cancer 74; 745-752

Andreansky et al 1996 PNAS 93; 11313-11318

Hunter et al 1999 J. Virol 73; 6319-6326

Stevens 1987 Science 235; 1056-1059

Samaniego et al 1998 J. Virol 72; 3307-3320

Krisky et al 1998 Gene Therapy 5; 1593-1603

Thomas et al 1999 J. Virol 73; 7399-7409

MacFarlane et al 1992 J. Gen. Virol. 73; 285-292

Howard et al 1998 Gene Therapy 5; 1137-1147

Wagstaff et al 1998 Gene Therapy 5; 1566-1570

Gossen M and Bujard H, 1992, PNAS 89: 5547-5551.

Gossen 1995, Science 268: 1766-1769.

Thompson et al, 1998 Virus Genes 1(3); 275-286

Meignier et al, 1988 J. Infect. Dis. 159; 602-614

1. A herpes virus strain capable of replicating in permissive cells which comprises a mutation which results in enhanced ICP0 expression compared to an otherwise identical virus without said mutation, for use in a method of treatment of the human or animal body therapy.

2. A virus strain according to claim 1 for use in a method of treatment of cancer.

3. A virus strain according to claim 1 or 2 which is capable of replicating in permissive cells to a greater extent than the otherwise identical virus strain without said mutation.

4. A virus strain according to any one of the preceding claims wherein said mutation comprises an alteration to the LAT regions of the virus.

5. A virus strain according to any one of claims 1 to 3 wherein said mutation comprises an alteration to the promoter region of the gene encoding ICP0.

6. A virus strain according to any one of the preceding claims wherein the ICP0 gene is operably linked to a non-ICP0 promoter.

7. A virus strain according to any one of the preceding claims which further comprises a mutation in a gene encoding ICP34.5, ICP6, glycoprotein H or thymidine kinase.

8. A virus strain according to any one of the preceding claims wherein said mutation enhances the expression of VMW65 compared to the otherwise identical virus without said mutation.

9. A virus strain according to any one of the preceding claims which is incapable of expressing a functional ICP34.5 protein and/or a functional ICP47 protein.

10. A virus strain according to any one of the preceding claims which further comprises a heterologous gene.

11. A virus stain according to claim 10 wherein said gene is capable of modifying an immune response.

12. A virus strain according to claim 10 or 11 wherein said gene encodes GM-CSF.

13. A virus strain according to anyone of the preceding claims which is a strain of herpes simplex virus 1 or 2.

14. A virus strain according to any one of the preceding claims wherein said permissive cells are tumour cells and non-tumour cells are non-permissive cells.

15. Use of a virus according to any one of the preceding claims in the manufacture of a medicament for the treatment of cancer.

16. Use according to claim 15 wherein the medicament is for direct intratumoral inoculation.

17. A method of treating a subject suffering from cancer, which method comprises administering a therapeutically effective amount of a virus strain according to any one of claims 1 to 14.

18. A pharmaceutical composition comprising, as active ingredient, a virus strain of the invention and a pharmaceutically acceptable carrier or diluent.

19. An agent for treating cancer comprising a virus strain capable of replicating in permissive cells which virus strain comprises a mutation which results in enhanced ICP0 expression compared to the otherwise identical virus strain without said mutation.
